(19) United States
(12) Patent Application Publication
(10) Pub. No.: US 2020/0410841 A1
(43) Pub. Date: Dec. 31, 2020

(54) Wireless device hub assisting users in adjustable timed hand wash sequences with visual and audible indicators with a method of collecting and reporting data

(71) Applicant: Dustin Richard Grzegorzewski, Orlando, FL (US)

(72) Inventor: Dustin Richard Grzegorzewski, Orlando, FL (US)

(21) Appl. No.: 16/929,105

(22) Filed: Jul. 15, 2020

(51) Int. Cl. G08B 21/24; G16H 40/67; G16H 40/20; G06Q 10/06; G06Q 10/10; G06Q 30/00; G16H 50/70; G06K 19/07

(52) U.S. Cl. CPC G08B 21/245; G16H 40/67; G16H 40/20; G06Q 10/0635; G06K 19/0723; G06Q 30/0185; G16H 50/70; G06Q 10/06316; G06Q 10/105

(57) ABSTRACT

A method for visually and/or audibly assisting a participating user in a set sequence of hand wash steps, visually and/or audibly assisting with sequence durations of hand wash steps, and collecting and reporting a plurality of hand wash sequences. A wireless device hub will begin the visual and/or audible sequences once triggered by a participating user through a manual switch, such as a push button, or a trigger “key”, such as an RFID tag, which may be housed in and/or on a wearable and/or non-wearable article such as an RFID badge, watch, wristband, pendant, glasses, cell phone, RFID card, and so forth. The hub will collect a timestamp each time the hand wash sequence is triggered and wirelessly transmit data for collection. The hub will wirelessly send data, such as timestamps and URLs, using a tag, such as RFID and/or NFC tag, to the trigger “key” article upon triggering the sequence which will allow a compatible device of the participating user to receive that data.

### FIELD AND BACKGROUND OF THE INVENTION

The present invention, in some embodiments thereof, relates to a system and a method for encouraging effective hand washing habits that assists in mitigating a plurality of harmful contaminant risks and spread of those contaminants. More particularly, but not exclusively, to a system and a method for reporting personal hand washing habits, such as a set sequence of hand washing events and adjustable durations of those events in the sequence, of participating users.

In today's age of potential infectious risks transmitted through touch, there is a need to provide knowledge and tools to ensure proper hand wash practices take place in the workforce by employees and at public facilities by the general public. Many environments that are considered a high touch risk include businesses that have high levels of person-to-person contact. These businesses include, but are not limited to, the food and beverage industry, retirement or home care, hotel and lodging, cruise line industry, sporting events, and many other hospitality, consumer, retail, and entertainment related fields. These businesses are especially targeted due to the large number of external customers. There is now, more than ever, a need for these and other industries to take advanced precaution by providing tools and the knowledge for their employees and customers to wash their hands according to recommendations and guidelines set forth by public health officials.

In recent years, other devices have been developed to improve overall hygiene among hospital staff and caretakers. These devices, and its process, allows for organizations to take more control over their employee hand washing habits by providing knowledge and tools to better ensure the safety of their staff and customers.

For example U.S. Pat. No. 8,698,637, filed on Sep. 3, 2009 describes a system for using a wearable monitoring device which monitors personal hygiene compliance of users in a typical environment such as hospitals, care homes, and hospices, where careers, such as nurses, doctors and therapists should wash their hands regularly, in particular, when moving between treating different patients. Broadly, the present invention comprises a wearable device that monitors the user and their hand washing compliance while communicating with a device to assist in location tracking of the user. This location tracker enables the device to remind the user to wash their hands through audible, vibratory, and/or visual depending on the location they have entered, such as a new patient room or a restroom.

Another example is described in UK Patent Application No. GB 2417811A, filed on Aug. 12, 2005, that discloses hand washing detector device, suitable to be fitted to a person possibly on the hand or wrist, comprises a sensor for sensing a hand washing event and a timing module responsive to the sensor. The device may also include an alarm system wherein the timing module activates the alarm system when a first predetermined time limit is exceeded between hand washing events. The alarm may be audible, vibratory and/or visual and the sensor may detect the presence of water or any other chemical present during hand washing. The device may help in infection control by monitoring hand washing and reminding people to wash their hands regularly.

Another example is described in U.S. Pat. No. 5,952,924, filed on Dec. 4, 1997 describes a system for encouraging workers who work in a hygienically controlled area to wash their hands before exiting the location such as a restroom. Broadly, the present invention comprises a device located in the sanitation area for receiving at least a portion of the hands of the worker. A detector is associated with the housing. The detector detects whether or not the hands of the worker have been recently washed in response to the insertion of the hands of the worker into the device housing. A communication media is also operatively associated with the detector for outputting one of a sanitary signaling media in response to the detection that the hands of the worker have been washed and an unsanitary signaling media in response to the detection that the hands of the worker have not been washed.

In addition, systems and methods for monitoring hygiene standards compliance in a certain environment have also been developed. For example, International Application No. PCT/EP2006/062895, filed on Jun. 2, 2006 describes a system and a method of monitoring hygiene standards compliance in a medical facility in which there is provided a surveillance network having a monitoring unit and a plurality of network units. There may additionally be provided a plurality of fixed network units. The monitoring unit, mobile network units and fixed network units are connected by way of a wireless personal area network (WPAN), in this case a ZigBee network. Identification signals are sent from the mobiles network units to the monitoring unit and the monitoring unit stores the identification signals in memory and generates a hygiene standards compliance profile for an individual associated with a particular mobile network unit. The hygiene compliance profile may provide information relating to the number of times that a particular individual washed their hands to information regarding the patients that that individual came into contact with over the course of a shift. Reports on the behavior of individuals or groups of individuals may be generated.

### SUMMARY OF THE INVENTION

According to some embodiments of the present invention there is a method and system for assisting and encouraging improvement of the hygiene habits, such as hand washing, while collecting and reporting those hygiene habits. The method comprises a hub that houses a plurality of electronic parts which enables it to collect participating user data based on a manual trigger and/or trigger “key” and wirelessly transmit the collected data to a database. The device visually and/or audibly displays and/or indicates a sequence of hand wash steps for the user to follow. The sequences are adjustable timed sequences in durations set by the administrator, owner, and/or establishment. The reporting sequence steps known as “phases”, are “Wet Hands”, “Apply Soap”, “Lather/Scrub”, “Rinse”, and “Complete”. The device will trigger the sequence steps each time a trigger “key” communicates with the device unless the device is in the “Complete” phase. In the “Complete” phase a trigger “key” that matches the identification “key” that begins the sequence will end the visual and/or audible indicators and not begin a sequence. A non-matching identification “key” will begin a sequence in the “Complete” phase.

The sequence phases are as described:

“Wet Hands” refers to the sequence step of visually and/or audibly indicating to the participating user to apply water to their hands in order to wet their hands.

“Apply Soap” refers to the sequence step of visually and/or audibly indicating to the participating user to apply soap to their hands.

“Lather/Scrub” refers to the sequence step of visually and/or audibly indicating to the participating user to scrub and lather their hands together.

“Rinse” refers to the sequence step of visually and/or audibly indicating to the participating user to run water on their hands to remove any soap, suds, contaminants, and so forth.

“Complete” refers to the sequence step of visually and/or audibly indicating to the participant that all prior set sequences have been completed in order for the set duration by the administrator, owner, and/or establishment.

Optionally, the method further comprises transmitting information, such as a URL and timestamps, to the participating user with a device capable of receiving such information.

Optionally, the method comprises adjustable durations and phases. The administrator, owner, and/or establishment can adjust the durations to any length of time and phases can be omitted from the sequence.

Optionally, the method further comprises a safety metric and/or pass/fail certification of the establishment in relation to a plurality of users according to the sequence frequency and duration of each event in the sequence.

Optionally, the safety metrics are based on a schedule of the user. While the non-employee users are omitted from the safety metrics pass/fail certification.

Optionally, the device comprises a motion sensor, such as infrared (IR) sensors, allowing the user to use a gesture, such as waving, or passing their hand near the face of the unit to manually trigger the set sequence without a “key” such as an RFID tag.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

The present invention will be described in even greater detail below based on the exemplary figures. The invention is not limited to the exemplary embodiments. Other features and advantages of various embodiments of the present invention will become apparent by reading the following detailed description with reference to the attached drawings which will illustrate the following:

In the drawings:

FIG. 1 is a schematic illustration of a system for allowing data collection using a wireless device hub from a plurality of participating users. Participating users are categorized as three subcategories: “registered users” are users using registered tags, such as an RFID tag, that are usually employed by the entity but not limited to employment, “unregistered users” are users without a registered tag that are usually employed by the entity but not limited to employment, and “guest users” not using tags and/or using unregistered tags, such as a personal cell phone and so forth,

FIG. 2 is a schematic illustration of a wireless device hub designed to house the components of the wireless device hub used to both collect and transmit data from and/or to the participating user and run a sequence of timed visual and/or audio steps to the participating user,

FIG. 3 is a chart of a method for analyzing risk mitigation of all users and/or each participating user. FIG. 3 also is a chart of a method for database storage and aggregation of data collected;

### DESCRIPTION OF SPECIFIC EMBODIMENTS OF THE INVENTION

The present invention, in some embodiments thereof, relates to a system, a method, and device for capturing timestamps, visually and/or audibly aiding participating users in using and following a sequence of hand washing steps and, more particularly, but not exclusively, to a system and a method for assisting in the proper personal hygienic hand washing steps of participating users.

According to some embodiments of the present invention there are provided methods, systems, and a wireless device hub for collecting timestamps of sequences of one or more participating users as well as aiding in visual and/or audible indicators.

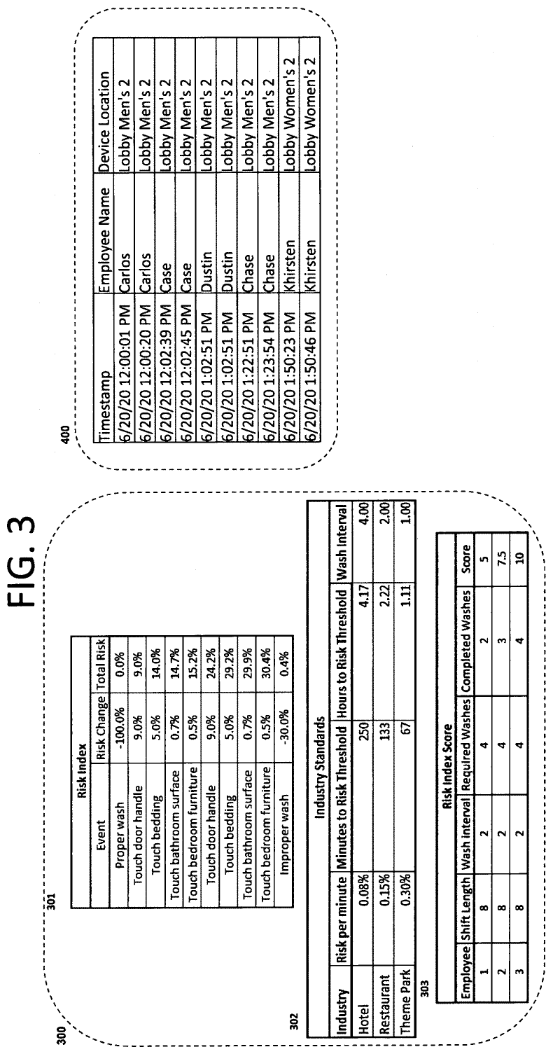

Optionally, the timestamps are collected and the data is aggregated into information that provides insight on risk reduction over the period of days, weeks, months, and/or years. Such embodiments allow scorecards on a plurality of users according to their timestamps that meet standards in their industry set by a risk index score shown in FIG. 3. These scores will be used to assist facilities such as restaurants, retail, hospitality, entertainments, and/or any company seeking to mitigate their risk index score, as further described below.

According to some embodiments of the present invention there is provided a method for collecting timestamps on participating users. There are two defined methods. Method 1 is based on a participating user with a registered trigger “key”, such as an RFID tag, that will communicate with the wireless device hub which will store a timestamp each time the registered trigger “key” communicates with the wireless device hub. The participating user must become a registered user by registering a trigger “key” via a registration terminal, such as a computer, mobile device, tablet, and/or other electronic device. The wireless device hub will temporarily store the identification of the registered trigger “key”, and transmit the data to the data cleaning process which will aggregate the data into the database for long term storage. Method 2 is based on a participating user without a registerable device, known as a “guest user” which may use an unregistered trigger “key”, such as an RFID tag that will communicate with the wireless device hub, and/or manual button on the wireless device hub. Each time the unregistered trigger “key” communicates with the wireless device hub and/or the manual button is depressed a timestamp is collected. The wireless device hub will temporarily store the timestamp of the unregistered trigger “key” and/or the manual button depress timestamp and transmit the data to the data cleaning process which will aggregate the data into the database for long term storage. The timestamp from the “guest user” will be aggregated into the database as a null and/or similar method, which will not be calculated into the risk index score. A “guest user” using a compatible device will receive stored information from the wireless device hub through a tag, such as an NFC and/or RFID tag, providing the “guest user” with promotional and/or informational data though methods such as an URL and/or QR code. As further described below, the database may be accessed via a communication network, such as the internet.

Optionally, the risk index score is based on the frequency and durations of participating users based on external data pertaining to that industry and risk of touchpoints, as further described below.

Optionally, the system ranks the risk index score and overall risk reduction of each one of the registered users with respect to the level of reduced risk indicated by the risk index score.

Before explaining at least one embodiment of the invention in detail, it is to be understood that the invention is not necessarily limited in its application to the details of construction and the arrangement of the components and/or methods set forth in the following description and/or illustrated in the drawings and/or the examples. The invention is capable of other embodiments or of being practiced or carried out in various ways.

### System

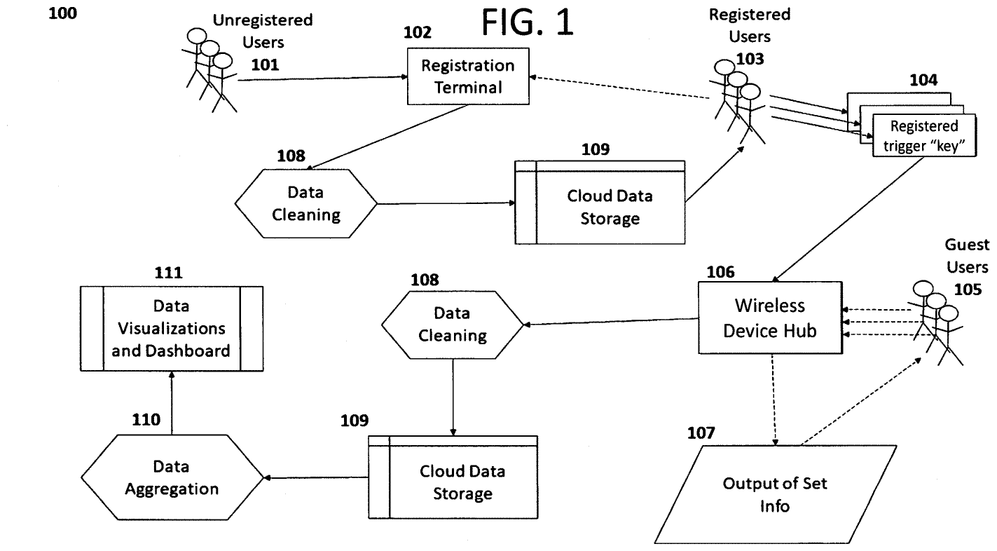

Reference is now made to FIG. 1, which is a schematic illustration of a system 100 for allowing data collection from a plurality of participating users using a registered trigger “key” 104, such as an RFID tag, for brevity these participating users will be referred to as “registered users” 103, and participating users using a manual trigger such as the manual switch and/or an unregistered trigger “key”, such as an RFID, for brevity these participating users will be referred to as “guest users” 105, according to some embodiments of the present invention. By collecting timestamps of registered users 103, analysis can be done to determine frequency and duration of sequences to improve registered user 103 habits and aid in sequence steps, and/or data can be collected from guest users 105 to better understand habits for marketing and educational purposes.

The system 100 is based on a wireless device hub 106, which collects a timestamp each time a registered user 103 or guest user 105 communicates with the hub using a registered trigger “key” 104 or manual trigger and outputs a set of information known as the output process 107 in the form of information such as a QR code, URL, etc. in which the guest user 105 may collect information using a plurality of compatible devices. The intent of the output process 107, is to allow entities to direct guest users 105 to their landing page in order to communicate brand standards, education on hygiene habits, safety precautions, etc. taken at their establishment. They may use this as a marketing platform to allow sign-up pages, loyalty pages, subscription pages, etc.

First-time and/or unregistered users 101 intending to become registered users 103 without a registered trigger “key” 104 or, optionally, registered users 103 needing a new registered trigger “key” 104 assignment will use the registration terminal 102, comprised of a plurality of electronic devices capable of network access such as a computer, mobile device, tablet, or other electronic device that is capable of connecting to the registration portal. The registration terminal 102 is comprised of a landing page allowing user input to assign their user identification, such as an employee identification number, to the registered trigger “key” 104. The registration terminal 102 will communicate to the database for data cleaning 108 which will assign the registered trigger “key” 104 to the registered user 103 to allow storage of the data in the cloud data storage 109.

Registered users 103 will use a registered trigger “key” 104 on the wireless device hub 106 to begin a sequence. The registered user 103 will be provided a visual and/or audible display of sequences to aid them in hand washing steps and durations. Once the sequence is complete, the registered user will use the registered trigger “key” 104 on the wireless device hub 106 to record a second timestamp. The first and second timestamp is used to determine the duration of the sequence of the registered user 103. The registered user 103 may choose not to complete the sequence, restart the sequence before the sequence is at the final step in the sequence, or restart the sequence after the completion step times out, in which the wireless device hub 106 will continue to record timestamps of the registered user 103.

The wireless device hub 106 will communicate and send the timestamps to the database cleaning 108 through a network, such as a wired network, for example, a local area network (LAN) and wide area network (WAN), or a wireless network, such as a wireless LAN (WLAN) and/or wireless personal area network (WPAN), and so forth. The data cleaning 108 will prepare the data for cloud data storage 109, in which the data will be accessed for data aggregation 110 to be used in reports and data visualizations 111, such as dashboards.

In some embodiments of the present invention, the risk index score 303, referred to in FIG. 3, per registered user 103 will be stored in the cloud data storage 109 in which the data can be turned into information through the data aggregation 110 to produce a risk index score 303.

### Wireless Device Hub

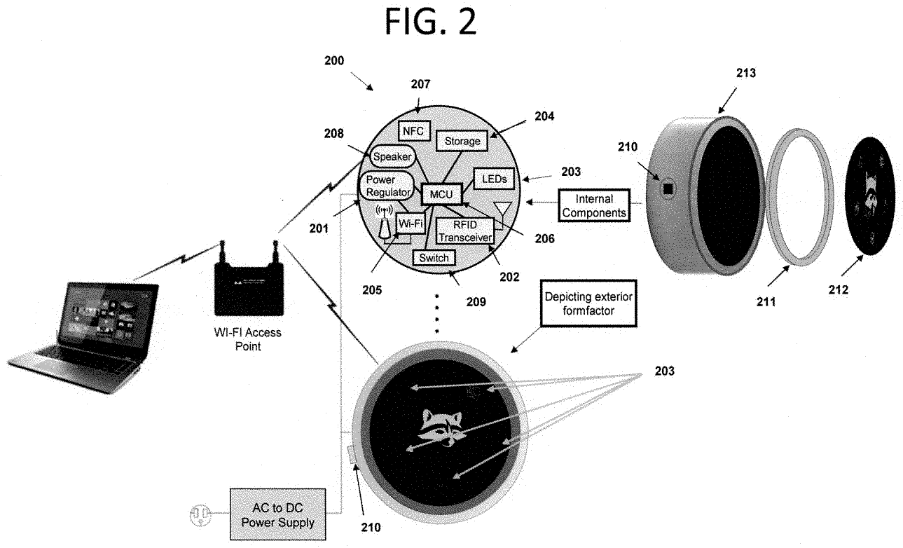

Reference is now made to FIG. 2, which is a schematic illustration of exemplary components of the wireless device hub 106 in FIG. 1, according to some embodiments of the present invention. FIG. 1 will be referenced throughout;

In some embodiments of the present invention, the wireless device hub 106 houses a printed circuit board (PCB) 200, a power regulator 201, a processor 206, such as a microcontroller which optionally may be connected to a Wi-Fi transmitter 205 and/or RFID transceiver 202, storage 204, a speaker 208, an NFC tag 207, an LED system 203, a manual switch 209, and a button 210.

In use, the RFID transceiver 202 is designed to detect the registered trigger “key” 104 and the processor 206 is designed to process and record the timestamp in the storage 204. In use, the processor 206, will “control” and process all functions, coding, calculations, sequences, and/or steps of all peripherals embedded on the PCB 200. In use, the Wi-Fi transmitter 205 will transmit data to the nearest connected Wi-Fi Access Point for data cleaning 108 and data storage in the cloud data storage 109. In use, the NFC tag 207 is designed to store and output data sets of information 107 to guest users 105. In use, the speaker 208 is designed to make a noise, tone, and/or chime to provide audio aids to registered users 103 and guest users 105. In use, the LED system 203 is designed to provide visual aid of sequence events and/or steps to registered users 103 and guest users 105. In use, the button 210 is designed to depress and/or trigger a manual switch 209 to begin the visual and/or audio sequences without the use of a registered trigger “key” 104. In use, the power regulator 201 is designed to supply power to the unit.

Optionally, the timed sequence indication is based on the related measurements. For example, the outer ring 211, may include one or more light emitting diodes (LEDs) which are designed to be activated during the timed sequence of a hand washing event and/or step. An LED color, such as red, is activated in response of failure to capture a complete timed sequence, for instance a failure to tap or reconnect the RFID from a registered user 103 and/or guest user 105 to the wireless device hub 106; an LED, such as green, is activated in response to a completed timed sequence, and any other variety of color combination activated for each stage of the timed sequence. In such an embodiment, the wireless device hub 106 provides the registered user 103 and/or guest user 105 with an immediate indication of the completion of the timed sequence of the hand washing event and/or step.

### Methods and Analysis

Reference is now also made to FIG. 3, which is a risk reduction method 300 for calculating a risk index score 303, of a registered user 103 in FIG. 1, according to some embodiments of the present invention. FIG. 1 will be referenced throughout. The risk index score 303 is a method, such as a calculation and/or equation, that measures risk reduction of the spread of infectious diseases, and/or other like risks, through surfaces with frequent touch points. The method that is described in FIG. 3 depicts a process of analyzing a risk index score 303 of a single registered user 103, and/or will be implemented simultaneously by any number of registered users 103, which will create a plurality of analytical metrics, such as averages and/or weighted scores for the entity as a whole. These metrics may be used to compare similar entities within an industry and/or unrelated industries and/or compare industries with other industries.

Now, as shown at 301, the risk index is formulated from external data calculating the +/−risk of events, such as touching certain surfaces, washing hands, washing hands improperly, and so forth. The equation may be calculated periodically, on schedule, or by request.

Optionally, the risk reduction method 300 takes into account the time of day during which the timestamps have been recorded and/or the frequency of the completed sequences. Optionally, these accounts of the time of day may be collected from the aggregated data, see data table 400. The account of the time of day during which the timestamps have been recorded and/or the frequency is captured in the risk index score 303 calculation. In such an embodiment, sequences missing after the working hours of the registered user 103 does not affect the risk index score 303.

In some embodiments of the present invention, the data is stored in the cloud data storage 109 to be aggregated 110 to one and/or more data visualizations and/or dashboards 111. The data visualizations and/or dashboards 111 of the registered user 103, and risk index scores 303 may be aggregated for the supervisor.

In some embodiments of the present invention, the data visualizations and/or dashboards 111 may present the risk index score 303 of one or more registered users 103 in relation to the risk index score 303 of other registered users 103. For example, the report may show the rank, leaderboard, scores, improvements, declines, and so forth by using the risk index score 303 of one and/or more registered users 103 in relation to the risk index score 303 of other registered users 103. In such an embodiment, the system 100 may be used to identify who are the registered users 103 with the highest and/or lowest risk index scores 303, and/or show which registered user 103 mitigated the most risk. As described above, the cloud data storage 109 may be accessed by remote clients via the data visualizations and/or dashboards 111, allowing remote users, such as administrators and managers to aggregate data 110 to issue the aforementioned reports and/or to search for current information that is related to registered users 103.

Optionally, the cloud data storage 109 is connected to a web server that hosts a designated website that allows permission access.

### Workers Management

In such an embodiment, the risk index score 303 of a worker during the time spent in work may be monitored and optionally rated. The arrival and departure times are used as anchors for the risk index score 303. The unregistered user 101 will use the registration terminal 102 at the beginning and end of their working shift to register the trigger “key”, such as an RFID, at the beginning of their shift, and unregister the trigger “key” at the end of their working shift. This will provide overall working hours which provides allotted amount of time the registered user 103 accumulates risk.

### System Management

For instance, FIG. 3, data table 400 depicts an exemplary dataset of logged timestamps related to sequences on the wireless device hub 106 in FIG. 1. FIG. 1 and FIG. 3 will be referenced throughout.

In use, the cloud data storage 109 manages a plurality of registered user 103 profiles for a plurality of participating registered users 103 and/or may be used for analyzing a risk index score 303 of said users.

Optionally, the risk index score 303 is used for reporting the risk mitigation of a group of registered users 103, which may form an entity. The risk index score 303 of each registered user 103 is optionally a relative rank that is determined according to timestamps of other registered users 103. For example, the risk index score 303 of each registered user 103 may be converted to a rank based on 1 to 10 that is set as the completion of a satisfactory amount of sequenced cycles.

It is expected that during the life of a patent maturing from this application many relevant apparatuses, methods, and systems will be developed and the scope of the terms personal hygiene sequence and wireless device hub is intended to include all such new technologies a priori.

As used herein the term “about” refers to .+−.10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for visually and/or audibly assisting users of sequenced hygiene habits while collecting data on their hygiene habits, comprising:
receiving, from an electronic device, hub, with set hygiene sequences related to the participating user, the plurality of personal hygiene sequences based on the manual and trigger “key” sequence starters;
recording a timestamp of each of the plurality of personal hygiene sequences based on the initiation of the sequence steps;
generating timestamp data for a database by logging said plurality of personal hygiene sequences and duration of each of the plurality of personal hygiene sequences;
calculating a safety and/or risk metric of the establishment based on the frequency and duration of the plurality of employed participating user hygiene sequences.

2. The method of claim 1, wherein said calculating the safety and/or risk metric is based on a schedule of the user.

3. The method of claim 1, wherein said collecting the user hygiene sequence data comprises recording a timestamp with user identification, such as an RFID tag.

4. The method of claim 1, wherein said generating the user hygiene profile comprises associating a timestamp with each personal hygiene event of the plurality of personal hygiene events, and wherein the method further comprises identifying at least one personal hygiene pattern according to respective said associated timestamps, and wherein said calculating the hygiene level is performed according to said at least one personal hygiene pattern.

5. The method of claim 1, further comprising:
receiving, from the hub, timestamp data regarding the user; and
identifying at least one employed user hygiene pattern according to said timestamp data, wherein said calculating the hygiene level is performed according to said at least one employed user hygiene pattern.

6. The method of claim 1, wherein said estimating the reduction of touch-related contamination transmission risk from each of the plurality of personal hygiene events is determined by measuring a member of a group consisting of: comparing the difference between user timestamps.

7. A electronic device hub for recording timestamps of hygiene habits of a participating user, comprising:
at least one sensor configured to record the presence of an RFID transmitter belonging to a participating user in which the electronic device records the timestamp;
a repository configured to visually and audibly assist the participating user in said plurality of personal hygiene sequence and/or sequences;
an interface configured to forward said recorded timestamps of personal hygiene sequences to a remote database, wherein said remote database is configured to:
maintain a company profile estimating the reduction of touch-related contamination transmission risk from each of the plurality of personal hygiene events by measuring participating users in a group and/or by comparing the difference between participating user timestamps; update said company profile according to said plurality of participating user hygiene sequences; and
calculate a unit measuring risk reduction of touch-related contamination transmission.

8. The hub of claim 7, further comprising a transmitter, such as an RFID tag and/or NFC tag, configured to send data such as timestamps and URLs to the trigger “key” article upon beginning the sequence for the participating user to collect.

9. The hub of claim 7, wherein said remote database is configured to:
maintain a company profile estimating the reduction of touch-related contamination transmission risk from each of the plurality of personal hygiene events by measuring participating users in a group and/or by comparing the difference between participating user timestamps;
update said company profile according to said plurality of participating user hygiene sequences;
calculate a unit measuring risk reduction of touch-related contamination transmission.

10. A system for reporting reduction of touch-transmitted contamination risk based on hygiene habits of a plurality of participating users, comprising:
a plurality of wireless electronic device hubs located near employee hand wash locations to allow recording and transmitting a plurality of participating user hygiene sequences related thereto, and a database configured to:
receive, from each of the wireless electronic device hubs, the plurality of personal hygiene sequences related to the respective participating user;
estimate the reduction of a touch-transmitted contamination risk unit based on each of the plurality of personal hygiene events for each of the plurality of participating users based on the timestamps received by the sensor;
generate a company risk reduction profile for each of the plurality of participating users by logging said plurality of personal hygiene sequences of each respective participating user and said risk reduction of each of the plurality of personal hygiene sequences of each respective user;
calculate a reduced risk of each of the plurality of users based on the user risk reduction profiles;
and rank the risk reduction of the plurality of users.
